(19) United States
(12) Patent Application Publication
(10) Pub. No.: US 2005/0246034 A1
(43) Pub. Date: Nov. 3, 2005

(54) Implantable mechanical device with adjustable geometry

(76) Inventor: Arnaud Soubeiran, Paris (FR)

Correspondence Address: SUGHRUE MION, PLLC, 2100 PENNSYLVANIA AVENUE, N.W., SUITE 800, WASHINGTON, DC, 20037, US

(21) Appl. No.: 10/525,653

(22) PCT Filed: Aug. 29, 2003
(86) PCT No.: PCT/FR03/02616

§ 371 Date: Feb. 25, 2005

(30) Foreign Application Priority Data
Aug. 30, 2002 (FR) 0210746

(51) Int. Cl. A61F 2/30

(52) U.S. Cl. 623/23.45; 623/23.47; 606/63

(57) ABSTRACT

The invention concerns an implantable mechanical device with adjustable geometry comprising, aligned and attached, an input components (1) having a first cylinder (11), an output component (2) having two second (21) and third (22) opposite cylinders, a reference component (3) integral in rotation with part of the organism and having a fourth cylinder (31). A friction spring (7) mounted astride on the first (11) and second cylinders (21) and a second friction spring (8), wound in an opposite direction of the spring (7) mounted astride on the third (22) and fourth (31) cylinders transform the reciprocating rotation applied to the input component (1) from outside the organism through means (931) into a rotation in a direction of the output component (2) which is helically connected (51, 52) to the driven component (4) integral with part of the organism by means (911), which is thereby translated. The device enables in particular manufacture of elongation nails and spiral rods.

The present invention relates to implantable devices with adjustable geometry inside the organism, such as bone-lengthening nails, expandable prostheses or spinal rods for distraction or compression, for example.

Several implantable mechanical devices with adjustable geometry exist, such as those described in documents US-A-5 074 882 or US-A-5 505 733 or US-A-4 892 546, which transform the rotation of one part relative to another into a lengthening, or FR 2 819 394, which transforms a shortening into a lengthening.

However, the existing devices are relatively complex and thus costly to produce; they are not easy to miniaturize and pose problems of fragility or medium- and long-term reliability, which limit their use.

The implantable mechanical device with adjustable geometry according to the invention proposes a simple and thus reliable solution, which is easy to produce and to miniaturize, in order to bring about a change in geometry within the organism. It comprises:
an input part having a first cylindrical end,
an output part, having two second and third cylindrical ends, which are opposite one another, and which are aligned, and where the second cylindrical end has a diameter equal to that of the said first cylindrical end,
a reference part having a fourth cylindrical end of the same diameter as the said third cylindrical end,
a transported part, having a helicoidal link with the said output part,
means to hold the axes of the said first, second and fourth cylindrical ends in alignment with the axes of the said helicoidal link, and to hold juxtaposed, firstly, the said first cylindrical end and the said second cylindrical end and, additionally, the said third cylindrical end and the said fourth cylindrical end, whilst allowing rotation of the said input part and of the said output part, relative to the said reference part, around the said axis of the said helicoidal link,
at least one first friction spring having an unloaded internal diameter slightly less than the common diameter of the said first and second cylindrical ends on which it is mounted by force, so as to straddle them,
at least one second friction spring wound in the opposite direction to the winding of the said first friction spring, and having an unloaded internal diameter slightly less than the common diameter of the said third and fourth cylindrical ends on which it is mounted by force, so as to straddle them,
means to bind the said transported part to a part of the organism,
means to apply to the said input part an alternative rotational movement from outside the organism,
means to block rotation of the said reference part relatively to a part of the organism.

The means for applying to the said input part an alternative rotational movement from outside the organism can, notably, be constituted by means to link respectively the said input part and reference part each to a different part of the organism, such as a bone or a segment of bone, able to be manipulated separately from outside the organism, which will be particularly advantageous in a bone lengthening nail or expandable prosthesis application, for example. They can also be constituted by one or more parts linked to the said input part or to the said reference part, placed under the skin or more generally in the soft tissues, and able to be manipulated through them in one direction at least, where the other direction can be achieved by elastic means which will have been compressed during handling in the first direction, for example, or by other parts also placed in the soft tissues in opposition with the first ones. The invention, its operation and its applications will be better understood, and others of its characteristics and advantages will be revealed, during the following description made on sight of the illustrations annexed for illustrative purposes, but by no means on a limitative basis, in which:

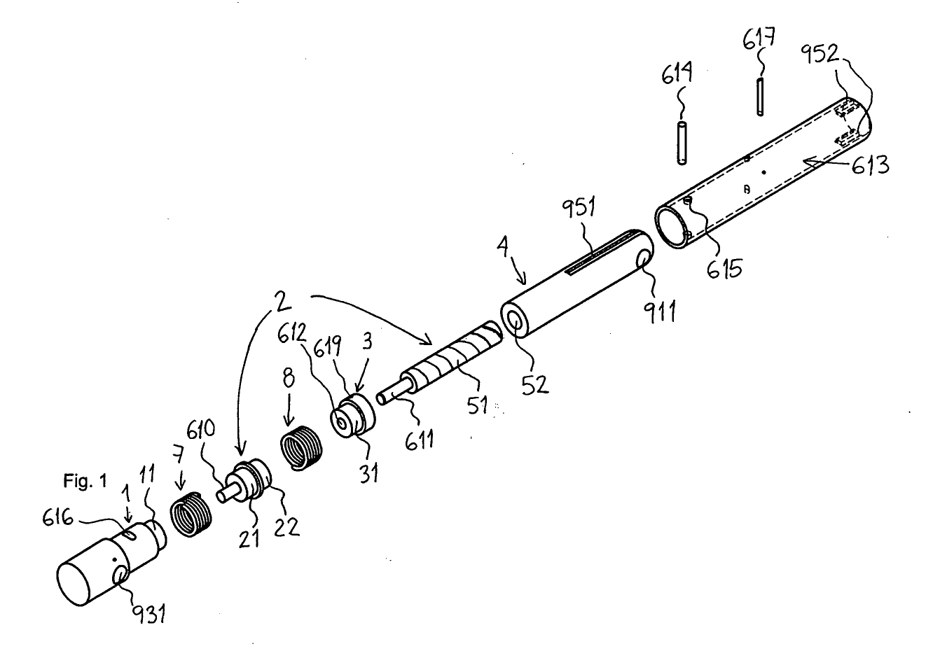

FIGS. 1 to 3 represent a preferred embodiment of the device according to the invention, which is particularly useful for limb lengthening.

FIG. 1 is an exploded perspective view of this embodiment;

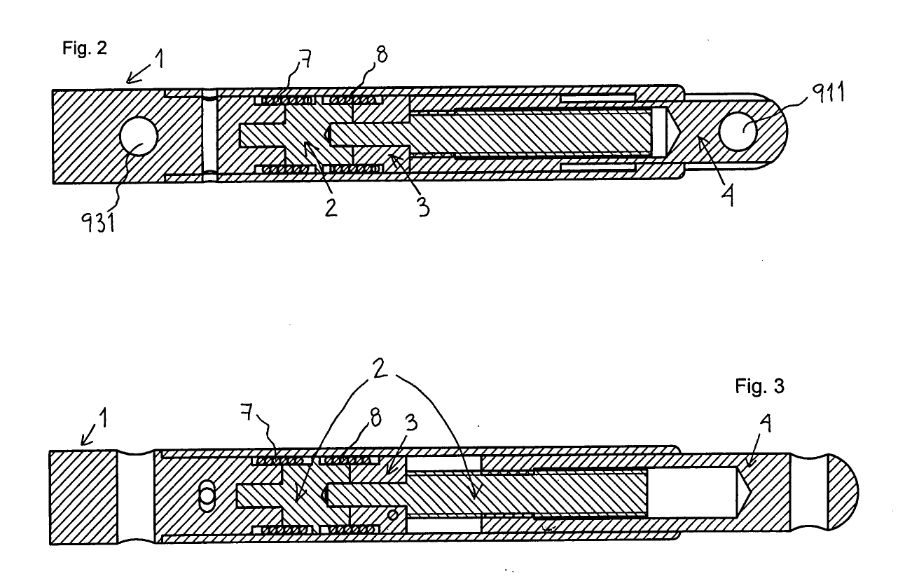

FIG. 2 is a cross-section view through the axis of the helicoidal link in its initial position;

FIG. 3 is a cross-section view, once again through the axis of the helicoidal link, but at 90° from that of FIG. 2, in which the said embodiment is represented partially lengthened;

FIGS. 4 to 6 represent a second preferred embodiment of the device according to the invention, which is particularly useful notably for spinal or intercostal distraction or compression;

FIG. 4 is an exploded perspective view of this second embodiment;

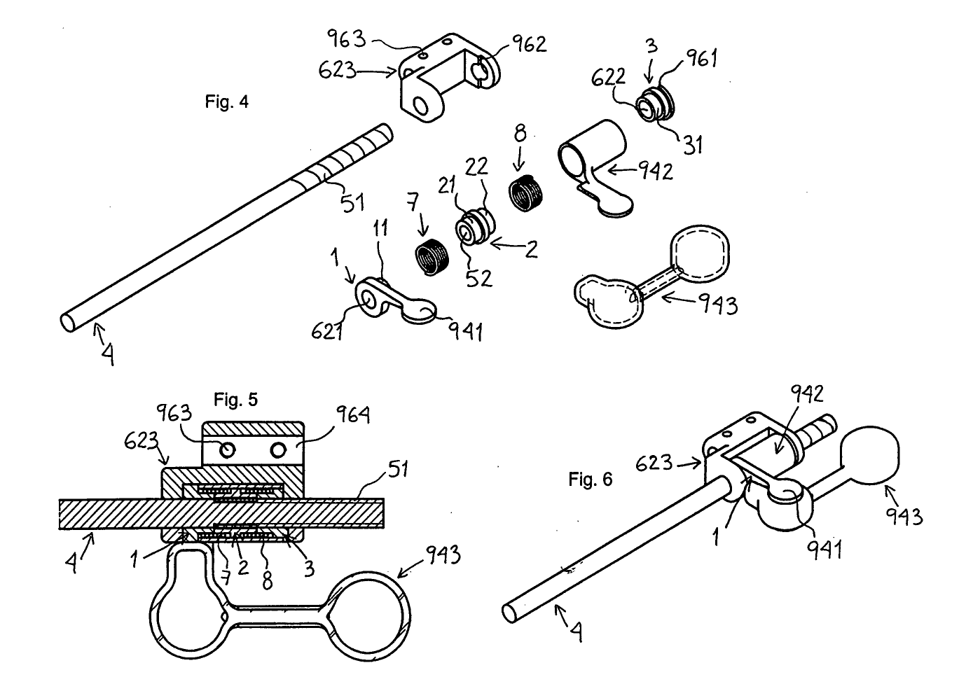

FIG. 5 is a partial cross-section view through the axis of the helicoidal link; and

FIG. 6 is a perspective view of this second embodiment, when assembled.

It is specified that, in these figures, the same references designate the same elements, whatever the figure in which they appear, and whatever the form of representation of these elements. Similarly, if elements are not specifically referenced in one of the figures, the reference can easily be found by referring to another figure.

In all the figures the hidden lines are invisible, except in the case of parts for which they facilitate understanding. In all the figures the dimensions and proportions have been altered when this was able to facilitate understanding.

The applicant is also keen to specify that the figures represent several embodiments of the object according to the invention, but that other embodiments exist satisfying the definition of this invention.

He also specifies that when, according to the definition of the invention, the object of the invention comprises “at least one” element with a given function, the described embodiment may comprise several of these elements.

He also specifies that if the embodiments of the object according to the invention as illustrated comprise several elements with identical functions and if, in the description, it is not specified that the object according to the invention must in all cases comprise a particular number of these elements, the object of the invention may be defined as comprising “at least one” of these elements.

All embodiments of the device according to the invention include:
an input part 1 having a first cylindrical end 11,
an output part 2, having two second 21 and third 22 cylindrical ends, which are opposite one another, and which are aligned, and where the second cylindrical end 21 has a diameter equal to that of the said first cylindrical end 11,
a reference part 3 having a fourth cylindrical end 31 of the same diameter as the said third cylindrical end 22,
a transported part 4, having a helicoidal link 51, 52 with the said output part 2,
means to hold the axes of the said first 11, second 21 and fourth 31 cylindrical ends in alignment with the axes of the said helicoidal link 51, 52, and to hold juxtaposed, firstly, the said first cylindrical end 11 and the said second cylindrical end 21 and, additionally, the said third cylindrical end 22 and the said fourth cylindrical end 31, whilst allowing rotation of the said input part 1 and of the said output part 2, relative to the said reference part 3, around the said axis of the said helicoidal link 51, 52,
at least one first friction spring 7 having an unloaded internal diameter slightly less than the common diameter of the said first 11 and second 21 cylindrical ends on which it is mounted by force, so as to straddle them,
at least one second friction spring 8 wound in the opposite direction to the winding of the said first friction spring 7, and having an unloaded internal diameter slightly less than the common diameter of the said third 22 and fourth 31 cylindrical ends on which it is mounted by force, so as to straddle them,
means to bind the said transported part 4 to a part of the organism,
means to apply to the said input part 1 an alternative rotational movement from outside the organism,
means to block rotation of the said reference part 3 relatively to a part of the organism.

Indeed, in the first preferred embodiment of the invention represented in FIGS. 1 to 3, the parts are assembled inside a tube 613 fitted at one end with internal protrusions 952, and at the other end with a first hole 615 perpendicular to its axis and, at some distance, with a second hole 618 perpendicular to its axis but non-convergent.

Input part 1 consists of three aligned cylinders having decreasing diameters. The one having the smallest diameter constitutes the said first cylindrical end 11 and is moreover perforated in its centre, to receive a centering pin, and the one having the largest diameter constitutes the other end which has at least one hole 931 perpendicular to its axis, through which a screw, which is unrepresented, can link the said input part 1, for example to a bone segment, thus constituting means to apply an alternative rotational movement from outside the organism. The intermediate cylinder has a diameter slightly less than the internal diameter of the said tube 613 inside which the said input part 1 is lodged and thus guided, relative to the said tube 613, and has an oblong hole 616 which is substantially perpendicular to its axis which, on assembly, is located facing the said first hole 615 of the said tube 613 which receives, when mounted by force, a pin 614 which, conversely, remains free to oscillate in the said oblong hole 616, thus giving the said input part 1 rotational freedom in both directions relative to the said tube 613 and giving the same to the parts which are linked rigidly to it under rotation.

Output part 2 consists of two parts 21, 22 and 51, 611. The first part comprises two second 21 and third 22 aligned cylindrical ends, for example, but not necessarily, separated by a shoulder of diameter less than the internal diameter of the said tube 613. The second cylindrical end 21 also receives in its extension a centering pin 610 aligned with it, of diameter only slightly smaller than that of the hole made to receive it in the said first cylindrical end 11 of the said input part 1 and the said third cylindrical end 22 receives an aligned hole which is used for assembling the two parts constituting the said output part 2. The second part of the said output part 2 consists of a centering and assembly pin 611 of a diameter slightly greater than the hole made in the said third cylindrical end 22 and a screw 51 aligned together. The two parts constituting the said output part 2 are assembled for example by hooping of the said centering and assembly pin 611 in the hole created to receive it in the said third cylindrical end 22. They fit tightly round, firstly, the said reference part 3, which is traversed by the said centering and assembly pin 611 through a hole 612 of diameter slightly greater than the said centering and assembly pin 611 to allow the rotation of the output part 2 and reference part 3 relative to one another and, secondly, the said second friction spring 8, which is previously mounted on the said third 22 and fourth 31 cylindrical ends.

The said reference part 3 consists of a fourth 31 cylindrical end extended by a shoulder of diameter slightly less than the internal diameter of the said tube 613 which comprises a hole 619 which on assembly is aligned with the said second hole 618 of the said tube 613 and receives a pin 617, assembled by force.

The said transported part 4 is substantially a cylinder rounded at one end to allow easy penetration in the organism, and comprising at the other end a concentric tapping 52 which constitutes, with screw 51 of the said output part 2, the helicoidal link between the said transported part 4 and said output part 2. The said transported part 4 also comprises at its end a hole 911 which is not parallel to its axis and which is able, for example, to receive a screw connecting to a bone segment, which is unrepresented, which constitute means to bind it to a part of the organism. They also comprise on their surface anti-rotation grooves 951 able to cooperate with the internal protrusions 952 of the said tube 613 to block the rotation between the said transported part 4 and the said tube 613. The said anti-rotation grooves 951 are generally rectilinear and parallel to the axis of the helicoidal link 51, 52, but can also be slightly helicoidal, if it is desired to have simultaneously a lengthening and the correction of a twisting, for example.

The said first friction spring 7 wound in the direction opposite that of the said second friction spring 8 is mounted by force so as to straddle them on the said first 11 and said second 21 cylindrical ends which are centered by means of the said centering pin 610 of the said output part 2.

Assembly of the first preferred embodiment of the invention is thus undertaken, for example, in the following order: one commences by assembling the said output part 2 by tightening the said reference part 3 and the said second friction spring 8 as indicated above. The transported part 4 is then screwed on screw 51 of output part 2; the first friction spring 7 is then assembled so as to straddle them on the said first 11 and said second 21 cylindrical ends, which also joins the said input part 1 to the previously constituted sub-assembly. The whole assembly is then introduced, with the rounded end of the transported part 4 first, in the end receiving holes 615, 618 of tube 613, and pushed into the said tube 613 until the anti-rotation grooves 951 of the said transported part 4 engage in the internal protrusions 952; the manipulations are then continued until the hole 619 of the shoulder of the said reference part 3 and the oblong hole of the said entry part 1 are successively aligned with the corresponding holes 618, 615 of the said tube 613 and by introducing by force pins 614, 617, which completes the assembly of the said first preferred embodiment of the invention.

Thus, in the first preferred embodiment of the invention, the means to hold the axes of the said first 11, second 21 and fourth 31 cylindrical ends aligned with the axis of the said helicoidal link 51, 52 are thus constituted essentially by the centering pin 610 and the centering and assembly pin 611 of the said output part 2, and the holes 612 with which they cooperate. Those to maintain juxtaposed the said first 11 and second 21 cylindrical ends, firstly, and the said third 22 and fourth 31 cylindrical ends, secondly, are essentially constituted by the said tube 613, the assembly pins 614, 617 and holes 615, 616 and 618, 619 which receive them.

The said reference part 3, in this first preferred embodiment of the invention, is, for its part, bound in rotation with a part of the organism by means of tube 613 to which it is linked by pin 617 and of the said transported part 4, which is blocked in rotation relative to the tube 613 by means of the anti-rotation grooves 951, and of the internal protrusions 952, and relative to a part of the organism by means of hole 951, which is able to receive, for example, a screw connecting to an unrepresented bone segment.

The operation of this first preferred embodiment of the invention represented in FIGS. 1 to 3 is simple: alternative rotations are applied to the input part 1 from outside the organism by twisting the limb containing the bone segments to which are respectively connected the said input part and the said transported part 4. When the rotation is applied in the opposite direction to the winding direction of the said first friction spring 7, the latter is tightened on the said first cylindrical end 11 and the second cylindrical end 21, which prevents it sliding, and a rotational movement is applied in its turn to output part 2. The second friction spring 8, which is wound in the opposite direction to the said first friction spring 7, slides, for its part, on at least one of the said third and fourth cylindrical ends and thus does not indeed oppose the rotation of the said output part 2. When the rotation is applied in the winding direction of the said first friction spring 7, the second friction spring 8 which is supported on the said reference part 3 opposes the rotation in this direction of the said output part 2, which thus remains in its angular position. Naturally, the rotation in the direction allowed by the said friction springs 7 and 8 of the output part 2 causes a translation movement in the direction depending on the direction of their helicoidal link 51, 52 of the said transported part 4. No translation movement in the reverse direction is possible.

In the second preferred embodiment of the invention represented in FIGS. 4 to 6, which is more particularly advantageous to undertake spinal or intercostal distractions or compressions but which is also of use with few modifications for the distraction of cranial or jaw bones, for example, the said transported part 4 is a cylindrical rod fitted with a threaded end 51 on which are inserted in order the said input part 1, the said first friction spring 7, the said output part 2, which is screwed on the said threaded end 51, the said second friction spring 8 and the said reference part 3. To allow their insertion, the said input part 1 and the said reference part 3 comprise respectively an emerging hole 621 according to the axis of the first cylindrical end 11, and an emerging hole 622 according to the axis of the fourth 31 cylindrical end, of common diameters which are slightly greater than the external diameters of the threads of the threaded end 51 of the said transported end 4, to allow rotation of the parts relative to one another. For its part, the output part 2 has a coaxial tapping 52 with the said second 21 and third 22 cylindrical ends able to cooperate with the threaded end 51 of the said transported part 4, thus constituting their helicoidal link 51, 52. Their structures, moreover, differ little from those of the corresponding parts of the first preferred embodiment of the invention.

The said transported part 4 and the holes 621, 622 and the threading 52 with which it cooperates thus constitute in this second preferred embodiment of the invention the means to hold the axes of the said first 11, second 21 and fourth 31 cylindrical ends aligned with the axis of the said helicoidal link 51, 52. Those to hold juxtaposed, firstly, the said first cylindrical end 11 and the said second cylindrical end 21 and, additionally, the said third cylindrical end 22 and the said fourth cylindrical end 31 are, for their part, constituted essentially by a U-shaped part 623 which holds them tightly between its branches and includes, close to the top of each of the branches of the said U, a hole of substantially the same diameter as those of the holes 621,6 122 of the said input part 1 and the said reference part 3 to be able to be inserted on the said transported part 4 with the parts which its encloses.

The means to bind the said transported part 4 to a part of the organism are, in a known manner, comprised of hooks, screws and other connectors, which are known but unrepresented in FIGS. 4 to 6.

The means to apply to the said input part 1 an alternative rotational movement from outside the organism are constituted by a lever 941 which is preferentially perpendicular to the axis of the helicoidal link 51, 52 and forming a single part with the said input part 1 which allows, by pressure through the soft tissues, the said input part 1 to be imparted with rotational movement in a first direction. The rotation of the said input part 1 in the opposite direction is then applied through a flexible pocket 943, for example made from silicon elastomer and possibly partially filled with physiological serum, a part of which is placed between a plate 942 which is relatively bound under rotation with the remainder of the device (in FIGS. 4 to 6 it also acts as a hood for parts of the device under rotational movement) and the said lever 941, and another part which communicates is positioned at a sufficient distance from the said lever 941 and pressed independently through the soft tissues, causing the reinflation of the part placed between the said plate 942 and the said lever 941 and thus the lifting of the said lever 941, which can then be depressed once again by pressure. The said plate 942 is not essential for the operation of the device if a hard part of the organism is located under the said flexible pocket 943. The said input part 1 can also be rotated in the opposite direction by elastic means, which are unrepresented, placed under the said lever 941, which have been compressed during the pressure exerted on the said lever 941, and these will tend to become relaxed once this pressure has disappeared. Advantageously the winding directions of the friction springs 7, 8 are chosen such that the pressure on the said lever 941 produces the rotation of the said output part 2 and that the means for return motion, whether elastic or using pressure on a part of the said flexible pocket 943, are used only to re-arm the device.

The means to bind in rotation the said reference part 3 relative to a part of the organism are constituted essentially by a plane 961 made in the said reference part 3 at the end opposite the said fourth cylindrical end 31, which cooperates with an additional plane made in the U-shaped part 623 which is connected to a vertebra, a rib or another part of the organism by means of rods, hooks and screws, familiar to those skilled in the art, which are themselves connected to the said U-shaped part 623 by means of a traversing hole 964 located at the base of the said U-shaped part 623 and substantially parallel with the said helicoidal link 51, 52, and of diameter suitable to allow the introduction of a known rod which will be held by screws placed in the tapping 963 made to this end, substantially perpendicular to the traversing perforation axis 964, also in a known manner. Alternatively, the U-shaped part 623 may comprise a hook-shaped part or other types of known connectors.

Having set out the specific operation of the means to impart to input part 1 an alternative rotational movement from outside the organism of the said second preferred embodiment of the invention, the remainder of the operation of the device may be deduced very easily from that of the first preferred embodiment of the invention to which reference should thus be made.

According to another embodiment, which is unrepresented, and which is of interest notably to produce an expandable component which is easily adaptable to every modular prosthesis system, the said transported part is a tube threaded on the inside with a helicoidal link with the said output part through a threading located between the second and third cylindrical ends of the said output part and of threading bottom diameter strictly greater than the external diameter of the friction springs mounted on the said cylindrical ends.

Among the advantages of the device according to the invention, the capacity to operate as a torque limiter of the friction springs may be observed, which allows, when this is desirable, the lengthening or compression force produced by the device to be limited to a given value, for example to accomplish a lengthening with constant controlled force instead of a lengthening with controlled displacement, which offers new clinical possibilities.

The device according to the invention may be made from all implantable materials which are sufficiently resistant for the envisaged application. Chromium-cobalt based alloys or high-performance polymers such as polyetheretherketone will notably be able to be used according to the application. The production of the device according to the invention is simple and will pose no problems for those skilled in the art.

The device according to the invention may advantageously be combined with various other means such as clutch means able to couple or uncouple the said input part from the means enabling a rotation to be applied to it, for example, or with itself, to obtain devices with a high level of control and possibilities for combined lengthening or shortening, for example.

The device according to the invention allows many implantable applications in the organism, among which bone-lengthening nails, particularly for the lower limbs, spinal or intercostal rods, for distraction or compression, devices for distraction of cranial or jaw bones, expandable prostheses or an expandable component able to be combined with all existing modular prosthesis systems, provided solely that the connectors are adapted to fit its ends.

1. An implantable mechanical device with adjustable geometry, comprising:
an input part (1) having a first cylindrical end (11),
an output part (2), having two second (21) and third (22) cylindrical ends, which are opposite one another, and which are aligned, and where the second cylindrical end (21) has a diameter equal to that of the said first cylindrical end (11),
a reference part (3) having a fourth cylindrical end (31) of the same diameter as the said third cylindrical end (22),
a transported part (4), having a helicoidal link (51, 52) with the said output part (2),
means to hold the axes of the said first (11), second (21) and fourth (31) cylindrical ends in alignment with the axes of the said helicoidal link (51, 52), and to hold juxtaposed, firstly, the said first cylindrical end (11) and the said second cylindrical end (21) and, additionally, the said third cylindrical end (22) and the said fourth cylindrical end (31), whilst allowing rotation of the said input part (1) and of the said output part (2), relative to the said reference part (3), around the said axis of the said helicoidal link (51, 52),
at least one first friction spring (7) having an unloaded internal diameter slightly less than the common diameter of the said first (11) and second (21) cylindrical ends on which it is mounted by force, so as to straddle them,
at least one second friction spring (8) wound in the opposite direction to the winding of the said first friction spring (7), and having an unloaded internal diameter slightly less than the common diameter of the said third (22) and fourth (31) cylindrical ends on which it is mounted by force, so as to straddle them,
means to bind the said transported part (4) to a part of the organism,
means to apply an alternative rotational movement to the said input part (1) from outside the organism,
means to block rotation of the said reference part (3) relatively to a part of the organism.

2. A device according to claim 1, wherein the means to apply to the said input part (1) an alternative rotational movement from outside the organism comprise means to link to at least one bone segment.

3. A device according to claim 1, wherein the means to apply an alternative rotational movement to the said input part (1) from outside the organism comprise means placed in the soft tissues.

4. A device according to claim 1, wherein the means to apply an alternative rotational movement to the said input part 1 from outside the organism comprise elastic means.

5. A device according to claim 2, wherein the means to apply an alternative rotational movement to the said input part 1 from outside the organism comprise elastic means.

6. A device according to claim 3, wherein the means to apply an alternative rotational movement to the said input part 1 from outside the organism comprise elastic means.
